(19) United States
(12) Patent Application Publication
(10) Pub. No.: US 2005/0266568 A1
(43) Pub. Date: Dec. 1, 2005

(54) Molecule-releasing apparatus and molecule-releasing medhod

(75) Inventors: Tsuyoshi Fujihara, Kawasaki (JP); Shozo Fujita, Kawasaki (JP); Shunsaku Takeishi, Kawasaki (JP); Kenji Arinaga, Kawasaki (JP); Yoshitaka Yamaguchi, Kawasaki (JP); Tatsuya Usuki, Kawasaki (JP); Ulrich Rant, Garching (DE); Karin Buchholz, Garching (DE); Marc Tornow, Garching (DE)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: FUJITSU LIMITED, Kawasaki (JP)

(21) Appl. No.: 11/101,658

(22) Filed: Apr. 8, 2005

Related U.S. Application Data
(63) Continuation of application No. PCT/JP03/07651, filed on Jun. 17, 2003.

(30) Foreign Application Priority Data
Oct. 10, 2002 (JP) 2002-297934

(51) Int. Cl. C12Q 1/68; A61N 1/30; C12N 15/87

(52) U.S. Cl. 435/459; 604/20

(57) ABSTRACT

A molecule-releasing apparatus includes a releasing unit configured to release molecules, wherein the releasing unit comprises at least one conductive member and releases molecules electrically interacting with the conductive member to which a potential is applied, from the conductive member by changing the potential so as to remove the interaction. In a moleculue-releasing method, molecules electrically attracted to two or more conductive members are released by electrical repulsion from the conductive members at different times. The molecule-releasing apparatus and molecule-releasing method are capable of efficiently releasing or transferring various useful molecules such as DNA molecules, into targets such as cells and can be safely applied for use in gene therapy and other applications.

### CROSS-REFERENCE TO RELATED APPLICATIONS

This is a continuation of Application PCT/JP2003/007651, filed on Jun. 17, 2003.

### BACKGROUND OF THE INVENTION

1. Field of the Invention

The present invention relates to a molecule-releasing apparatus and molecule-releasing method that are capable of efficiently releasing or transferring various useful molecules such as DNA molecules, into targets such as cells and can be safely applied for use in gene therapy and other applications.

2. Description of the Related Art

Gene therapy, although it is a revolutionary therapy, has been limited to the applications such as lethal monogenic diseases (e.g., severe combined immunodeficient, familial hypercholesterolemia) cancer, and AIDS (acquired immunodeficiency syndrome) for some reasons that there are few diseases of which involvements with genes have been elucidated, and that no safe and efficient transgenic method has been established. In 1995, Varmus et al. of NIH (National Institutes of Health) reported that no disease had been cured by genes transferred in gene therapy. However, in light of the completion of the full human genome sequence by the Human Genome Project in April 2003, if the further developments in functional genome science and structural genome science elucidate the involvement of the sites on the decoded human genome sequence with the expression of functions, the applications of the gene therapy would be extended to treatment of various diseases including lifestyle-related diseases (e.g., diabetes) that had not been treated by the therapy.

Gene transfer is essential in the gene therapy, but until today no safe and efficient transgenic method has been established. Actually it was reported that in September 1999, an eighteen-year-old young man died four days after he received gene therapy at Pennsylvania University in the U.S.A. because of an adenovirus vector used to mediate gene transfer and administered in excess of a prescribed dose. In light of these situations, it is strongly needed to establish a safe and efficient non-virus vector-mediated gene transfer method.

The non-virus vector-mediated transgenic method includes the vesicular liposome method, the gene gun method, and the microinjection method.

Of these methods, the vesicular liposome method has been most frequently used as a non-virus vector for its low antigenicity (that suppresses immune responses), capability of transferring large genes, and high transfer efficiency. However, under the vesicular liposome method, injected genes are not transferred into genomes, expressions are transient and are lowered particularly in nondividing cells.

The gene gun method is a technique to physically and forcibly transfer genes into cells as if to bombard them with a gun (Japanese Patent Application Laid-Open (JP-A) No. 05-68575 and JP-A No. 2000-125841). However, under the method, genes or particles are bombarded into cells, causing problems of damages to cells, inadequate safety and high energy cost. On the other hand, there is a disclosed method of binding DNA molecules to a gold electrode via thiol groups and releasing the DNA molecules from the substrate by applying a negative potential to the gold electrode (J. Wang et al., Langmuir, 15, 6541-6545 (1999)). However, the method requires much energy to decompose the thiol groups for releasing the DNA molecules, causing the problems of cost and safety.

The microinjection method is considered as the most reliable method to transfer genes to cells. However, under the method, a DNA solution is directly injected into cells using a hollow needle of which size is large in comparison with the cell size, which greatly damages the cells and requires cell fixation. At present the fixation of somatic cells with a vulnerable cell membrane is impossible, thus the injected genes are not transferred into the genomes and the expression is transient.

Recently, it was reported that under the recombinant pre-integration complex (rPIC) method, in which a complex formed just before a recombinant virus that has infected a cell and entered the nucleus are integrated into the genome (recombinant pre-integration complex; rPIC) is previously prepared and transferred into a target cell by an appropriate means such as micromanipulator, genes can be transferred into the genome of the target cells irrespective of the species of the target cells and the expression is persistent (TAKARA HOLDINGS INC., “Development of Recombinant Pre-Integration Complex (rPIC) Method” [on-line], Dec. 11, 2001 [Searched on Apr. 21, 2003], Internet <URL: http://www.takara.co.jp/news/2001/10-12/01-1-037.htm>). However, the method is insecure about safety as it is not regarded as a non-virus vector-mediated method, and the preparation of the rPIC requires much cost and time.

Thus, in the gene therapy, at present there is no technique that can efficiently, cost-effectively, and safely transfer molecules such as genes into target cells in desired positions at a desired time.

An object of the present invention is to provide a molecule-releasing apparatus and molecule-releasing method that resolves conventional problems, and are capable of efficiently releasing or transferring various useful molecules, e.g., DNA molecules, into targets such as cells without damaging them. The present invention can be safely applied for use in gene therapy and other applications.

### SUMMARY OF THE INVENTION

The present invention provides a molecule-releasing apparatus including a releasing unit configured to release molecules, wherein the releasing unit includes at least one conductive member and releases molecules electrically interacting with the conductive member to which a potential is applied, from the conductive member by changing the potential so as to remove the interaction. In the molecule-releasing apparatus, the releasing unit configured to release molecules causes the molecules electrically interacting with the conductive member to which a potential is applied, to be released from the conductive member by changing the potential so as to remove the interaction. At this moment, if the transfer targets of the molecules are arranged in proximity of the conductive member, the released molecules are transferred into the targets such as cells.

The present invention further provides a molecular releasing method wherein molecules electrically attracted to two or more conductive members are released by electrical repulsion from the conductive members at different times. Under the method, the molecules are released at different times by electrical repulsion by changing the potential on (applying a voltage to) the two or more conductive members at different times, and transferred into targets such as cells.

Further objects, features and advantages of the present invention will become apparent from the following description of the preferred examples with reference to the attached drawings.

### BRIEF DESCRIPTION OF THE DRAWINGS

FIGS. 1A and 1B are schematic illustrations representing an example of the principle of releasing molecules in the molecule-releasing apparatus of the present invention.

FIG. 2 is a schematic illustration representing an example of the condition where molecules are electrically attracted to an electrode in the molecule-releasing apparatus of the present invention.

FIG. 3 is a schematic illustration representing an example of the condition where the molecules shown in FIG. 2 are released from the electrode by electrical repulsion.

FIGS. 4A, 4B and 4C are schematic illustrations for describing an example of the process of releasing a molecule from an electrode and transferring the molecule into a cell.

FIG. 5 is a schematic illustration representing an example of the relationship between the voltage applied to electrodes and the release of DNA molecules (those weakly interacting with the electrodes).

FIG. 6 is a schematic illustration representing an example of the relationship between the voltage applied to electrodes and the release of DNA molecules (excluding those weakly interacting with the electrodes).

FIG. 7 is a schematic illustration representing an example of the relationship between the voltage applied to electrodes and the release of DNA molecule-protein complexes.

FIG. 8 is a schematic illustration representing an example of a bed-of-nails electrode substrate on which many needle electrodes are arranged.

FIG. 9 is a schematic illustration representing an example of the monolayer culture of normal human dendritic cells.

FIG. 10 is a schematic illustration representing an example of the condition where a needle electrode is stabbed into a cell.

FIG. 11 is an enlarged schematic illustration representing an example of the condition where a DNA molecule is transferred into the cell shown in FIG. 10.

FIG. 12 is a schematic illustration representing an example of the condition where a cell is stabbed by a needle electrode and second electrode.

FIG. 13 is an enlarged schematic illustration representing an example of the condition where a DNA molecule is transferred to the cell shown in FIG. 12.

### DESCRIPTION OF THE PREFERRED EMBODIMENTS

(Molecule-Releasing Apparatus)

The molecule-releasing apparatus of the present invention comprises a molecule-releasing means and may further comprise any other means such as a monitoring means suitably selected according to necessity.

The molecule-releasing means has a function to release the molecules electrically interacting with at least one conductive member to which a potential is applied, from the conductive member by changing the potential so as to remove the interaction.

The conductive member(s) is not specifically limited as long as it is conductive, preferred examples include electrodes, and its shape, structure, size, surface property, number, material and other properties can be appropriately selected according to the purpose. Hereinafter the electrodes may be referred to as molecule-releasing electrodes.

The shape includes plate shape, needle shape, stick shape and spherical shape. These shapes may be used alone, or two or more may be used in combination. Among them, needle shape (needle electrode) is preferred for the purposes such as gene therapy where the molecules are directly released or transferred to the transfer targets of the molecules such as cells.

The structure may be comprised of single member or two or more members. In the former, the member must be conductive. In the latter, at least one member must be conductive, and such structures include a substrate and a layer of the material (conductive layer) provided thereon. The substrate is not specifically limited, its structure and size can be suitably selected, and its shape includes plate shape, needle shape, stick shape, and spherical shape. Its material is preferably conductive or insulating, and the insulating materials include silica glass, silicon and silicon oxide.

The size is, for example, about 500 μm or less in width or diameter, and preferably equal to or less than 300 μm in width or diameter when the molecule-releasing apparatus is manufactured in chip form, and preferably equal to or less than 100 μm when manufactured in fine chip form. When the electrodes are needle electrodes, in light of applications such as gene therapy, the diameter is preferably equal to or less than 0.2 μm and the length is preferably equal to or more than 3 am.

The number may be, for example one, or may be two or more. In light of the efficiency of releasing the molecules, the number is preferably two or more.

When the number is two or more, the material, shape, structure, size and surface property of the electrodes may be identical, or may be different with one another.

The arrangement of the two or more electrodes is not specifically limited, can be selected according to the purpose, and may be arbitrary, or may be orderly. In the latter, the electrodes can be equally spaced out.

The space between the two or more electrodes can be selected according to the size of the transfer targets of the molecules such as cells, for example the size of cells that have been monolayer cultured on a plate (approximately 10 μm), and is preferably about 1-5 μm when the molecules are transferred (introduced) to the cells that have been monolayer cultured on the plate.

Control (change of potential, application of voltage) of the two or more electrodes may be carried out at the same time, or may be carried out at the different times with one another.

The two or more electrodes may hold either of the same molecules or different molecules. In the latter, plural kinds of the molecules such as genes can be efficiently released or transferred to the transfer targets of the molecules such as cells.

When the two or more the electrodes are the needle electrodes, the two or more needle electrodes are preferably arranged to stand on a substrate.

In such a case, if a plate is selected as the substrate, a bed-of-nails substrate can be fabricated. The bed-of-nails electrode substrate can, for example in gene therapy, efficiently release or transfer (introduce) the molecules to the targets of the molecules such as cells that have been monolayer cultured on a plate. On the other hand, if a fiberscope is selected as the substrate and the needle electrodes are arranged at the tip of the fiberscope, it is possible to move the needle electrodes to the target sites in a living body (e.g., cancer cells, affected organs) for paracentesis, bringing about advantages to gene therapy and other applications.

Examples of the material are metals, conductive resins, and conductive carbons. These may be used singly, or two or more may be used in combination. In the latter, for example the conductive resins or conductive carbons may be coated with the metals.

Examples of the metals are metals such as gold, platinum, silver, copper and zinc, and their alloys.

Examples of the conductive resins are polyacetylene, polythiophene, polypyrrole, poly-p-phenylene, polyphenylene vinylene and polyaniline.

Examples of the conductive carbons are carbon nanotubes and carbon nanohorns. Examples of the carbon nanotubes are single-wall carbon nanotubes (SWNTs) and multi-wall carbon nanotubes (MWNTs), and specific examples of the single-wall carbon nanotubes are armchair carbon nanotubes, zigzag carbon nanotubes, and chiral carbon nanotubes.

These may be used singly, or two or more may be used in combination. Among them, when the electrodes are needle electrodes, at least one of those selected from metals having nanopillar structure, needle-shaped conductive plastics and carbon nanotubes is preferred. The nanopillar structure is a structure in which a plurality of pillarets of the order of nanometers are arranged to project. The metals having nanopillar structure can be formed by etching the metals.

In the conductive members, the sites where the molecules are held by interaction are not specifically limited and can be suitably selected according to the purpose. For example, when the conductive members are the plate electrodes, the preferable site may be the plate surface of the plate electrodes, and when the conductive members are the needle electrodes, the preferable site may be at least the tip of the needle electrodes. When the conductive members are the needle electrodes, they have an advantage in efficiently releasing or transferring (introducing) the molecules to transfer target of the molecules such as cells in the following way. Specifically, under the condition that the molecules electrically attracted and held to the tip of the needle electrodes and the needle electrodes are kept to be stabbed into the transfer target of the molecules such as cells, the molecules are released from the needle electrodes.

The arrangement of the conductive member is not specifically limited and can be suitably selected according to the purpose. In light of the efficiency of releasing or transferring the molecules to the targets, the conductive members are preferably arranged, for example in proximity to the transfer target of the molecules, and when the conductive members are the needle electrodes, the conductive members are preferably arranged to be stabbed into the transfer target of the molecules. When the molecules are electrically attracted to the conductive members and the molecules are released from the conductive members by an electrical repulsion, the conductive members are preferably arranged in a conductive medium. The conductive medium is not specifically limited, can be suitably selected according to the purpose, and may be, for example, any of, liquid, solid, and mixture thereof. Examples of the liquid are water, ion solutions, and solutions containing electrolytes, and preferred examples are the cells in light of gene therapy. These may be used singly, or two or more may be used in combination.

The transfer targets of the molecules are not specifically limited and can be suitably selected according to the purpose. Preferred examples are cells and microcapsules. The cells are not specifically limited, and include animal cells, plant cells and microorganisms. Among them, animal cells such as human cells are preferred for allowing monolayer culture on a plate, and for allowing easy and efficient transfer of the molecules. When the targets are cells and the molecules are genes, the cells are preferably surface-treated in light of the efficiency of transferring the genes. The surface treatment is not specifically limited, can be suitably selected from publicly known methods such as calcium phosphate treatment in accordance with purposes. The molecule-releasing means may comprise at least one of second electrode. When the second electrode is provided, the second electrode forms a pair of electrode in combination with the conductive member (electrode) to thereby form an electrical circuit in combination with the conductive member (electrode). This allows electrical currents to be balanced. By suitably changing the potential (applying a voltage) between the second electrode and the conductive member (electrode), the molecules can be electrically released from or attracted to the conductive member (electrode). For example, when a positive potential is applied to the conductive member (electrode) and the molecules are attracted to the conductive member (electrode) by electrical interaction, the application of a negative potential to the conductive member (electrode) and the resulting change of the potential (applying voltage) enables releasing the molecules from the conductive member (electrode) by electrical repulsion.

The second electrode(s) is not specifically limited for its shape, structure, size, material, and other properties and can be suitably selected from publicly known electrodes such as those explained as the conductive member according to the purpose. Among them, needle electrodes in needle shape are preferred.

The number of the second electrode is not specifically limited, and can be suitably selected according to the purpose. The number is normally equal to or less than the number of the conductive member (electrode), and preferably as less as possible. For example, when the conductive members (electrodes) are arranged to stand on the substrates and the number of the substrates is two or more, the number of the at least one of second electrode is preferably one per substrate. In such a case, the conductive member (electrode) and the second electrode are preferably arranged to face each other on the substrate, wherein the second electrode may be arranged to stand on the substrate, wherein the second electrode may be arranged in the center of the substrate with the second electrode surrounded by the conductive members (electrodes), or lines in which the second electrodes are equally spaced and lines in which the conductive members are equally spaced may be alternately arranged.

The molecule-releasing means may have at least one of third electrode in addition to the second electrode. In such a case, control is carried out by so-called three-electrode method that can more easily control potential (reference potential) between the conductive member (releasing electrode) and the second electrode than the two-electrode method that does not use the third electrode. For example, the third electrode can be used as the electrode for measuring or observing the reference potential.

The third electrode(s) is not specifically limited for its shape, structure, size, material, and other properties and can be suitably selected from publicly known electrodes such as those explained as the conductive member according to the purpose.

The number of the third electrode is not specifically limited, and can be suitably selected according to the purpose. In general the number is preferably smaller, and, for example, when the conductive members (electrodes) are arranged to stand on the substrates and the number of the substrates is two or more, preferably one per substrate. In such a case, the conductive member (electrode) and the third electrode are preferably arranged to face each other on the substrate, wherein the third electrodes may be arranged to stand on the substrate, or the third electrodes may be arranged in the center of the substrate.

The molecule-releasing means may comprise a power source. The power source connected to the conductive member (electrode) and the second electrodes can form, in combination with the conductive member (electrode) and the second electrode, an electric circuit that forms an electric field. The formation of the electric field allows arbitrarily change of the potential on (application of voltage to) the conductive member (electrode). The electric field is not specifically limited, and may be a direct-current electric field, or may be an alternating-current electric field.

The molecules are not specifically limited for their shape and other properties as long as they contain at least a region that can electrically interact with the conductive member, and can be suitably selected according to the purpose. Examples of the shape are line shape, granular shape, plate shape, and the combination of two or more of them, and among them line shape is preferred.

The preferred examples of the region that allows electrical interaction include a region having electrical polarity.

The shape of the region is not specifically limited, and can be suitably selected according to the purpose. The examples of the shape include line shape, granular shape, plate shape, and the combination of two or more of them, and among them line shape is preferred.

The size of the region is not specifically limited, and can be suitably selected according to the strength and other properties of the interaction. In light of securely holding the molecules to and efficiently releasing from the conductive member, the size is preferably larger, and may cover the whole region of the molecules.

The number of the region may be one, or may be two or more per the molecule. In the latter, electrical forces of interaction (e.g., cohesive strength) between the region and the conductive member may be identical or different with one another.

The region may further partially include a site having different electrical force of interaction (e.g., cohesive strength) with the conductive member. For example, when the region is a polynucleotide, a (CH₂)₃SS(CH₂)₃OH group or the like can be introduced to the 3′ terminal of the polynucleotide, where the SS moiety of the group is more strongly bound to the conductive member (e.g., metal electrode). Thus, molecules partially having a site that electrically interacts with the conductive member more strongly are not released only by applying a low voltage to the at least one conductive member (when the variation in the potential is small). As a result, the molecules that can be released (molecules not having the group) from the conductive member conductive member by applying a low voltage (even when the variation in the potential is small) and the molecules that cannot be released (molecules having the group) unless applying a high voltage (unless the variation in the potential is high) can be formed. When the former is used, the molecules can be released from the conductive member (electrode) at low cost, and when the latter is used, the molecules can be released from the conductive member without being influenced by variations in environmental conditions and other factors.

The molecules are not specifically limited, and can be suitably selected according to the purpose. Preferred examples include ionic polymers capable of electrically interacting with the conductive member (e.g., by binding), and among them biomolecules are more preferred in light of applications such as treatment of diseases.

The ionic polymers are preferably selected from positive ion polymers and negative ion polymers.

Preferred examples of the positive ion polymers (positively charged ionic polymers) include guanidine DNA and polyamine.

Preferred examples of the negative ionic polymers (negatively charged polymers) include polynucleotide and polyphosphoric acid. They are preferred in allowing easy control of interaction (e.g., binding) with the conductive member due to the presence of negative charges throughout the molecules at regular intervals, and polynucleotide is particularly preferred in light of use in gene therapy and other applications.

Examples of the polynucleotide are genes and their complexes (complexes between genes and proteins).

Examples of the genes are cancer-related genes, genetic diseases-related genes, virus genes, bacterial genes, and polymorphic genes that are referred to as disease risk factor.

Examples of the cancer-related genes are k-ras gene, N-ras gene, p53 gene (lung cancer, esophageal cancer, liver cancer), BRCA1 gene, BRCA2 gene, src gene, ros gene, GM-CSF gene (kidney cancer, prostatic cancer), thymidine kinase gene (prostatic cancer), anticancer agent-resistant gene MDR1 (breast cancer), and APC gene.

Examples of the genetic diseases-related genes are those relating to various inborn errors of metabolism such as phenylketonuria, alkaptonuria, cystinuria, Huntington's chorea, Down's disease, Duchenne's dystrophy, hemophilia, severe combined immunodeficient, and familial hypercholesterolemia.

Examples of the virus genes and the bacterial genes are hepatitis C virus, hepatitis B virus, influenza virus, measles virus, HIV virus, mycoplasma, rickettsia, streptococcus, and Salmonella.

Examples of the polymorphic genes are genes whose individuals have different base sequences that are not necessarily directly related to the cause of diseases, such as PS1 (presenilin 1) gene, PS2 (presenilin 2) gene, APP (beta amyloid precursor protein) gene, lipoprotein gene, HLA (human leukocyte antigen), blood type-related gene, and genes regarded as related to the occurrence of certain conditions such as diabetes and hypertension.

Examples of the gene complexes are complexes between genes and proteins including enzymes such as integrase (recombinant Pre-Integration Complex; rPIC) that are formed immediately before a recombinant virus that has infected a cell and entered the nucleus are integrated into the genome. The DNA and RNA may be a single strand, and may be a double strand.

The preparation method for the polynucleotide is not specifically limited, and can be suitably selected according to the purpose from publicly known methods such as a method using a DNA synthesizer (DNA automatic synthesizer), and a method in which previously prepared oligonucleotide sequences are annealed with lined monomer blocks, and bound together by DNA ligase or RNA ligase.

The length of the polynucleotide is not specifically limited, and can be suitably selected according to the purpose. However, even in the antisense method that is expected to be effective with shortest strands, at least six bases are preferred in light of the stability of the double strand. In general, the shorter the polynucleotide, the lower the peak voltage required to release it from the conductive member (required variation in the potential is reduced).

The number of the kind of the molecules to be used is not specifically limited, and can be suitably selected according to the purpose. The number may be one, or may be two, and in the latter, plural kinds of the molecules can be released or transferred to the transfer targets of the molecules such as cells.

The molecules may have a label in light of easy determination whether they have been released from the conductive member.

Examples of the label are radioisotopes, chemiluminescent substances, fluorescent substances, enzymes, antibodies, and others.

Examples of the radioisotopes are ³²P, ³³P, and ³⁵S.

Examples of the chemiluminescent substances are acridinium ester, luminol, isoluminol, or derivatives thereof.

Examples of the fluorescent substances are fluorescent dyes such as fluorescein series, rhodamine series, eosin series, and NBD series; green fluorescent protein (GFP); rare-earth cahelates such as europium (Eu), terbium (Th), and samarium (Sm); tetramethyl rhodamine, Texas red, 4-methylumbelliferone, 7-amino-4-methylcoumarin, Cy3, and Cy5.

Examples of the enzymes are peroxidase, alkaline phosphatase, glucose oxidase, β-galactosidase, and luciferase.

Examples of the others are biotin, ligands, specific nucleic acids, proteins, and haptens. When the label is biotin, it can be combined with avidin or streptavidin that specifically bind to it; when the label is hapten, it can be combined with antibodies that specifically bind to it; when the label is a ligand, it can be combined with a receptor; and when the label is a specific nucleic acid, protein, hapten or the like, it can be combined with nucleic acids, nucleic acid-bound proteins, and proteins having affinity for specific proteins.

The molecule-releasing means is not specifically limited as long as it can release the molecules electrically interacting with the conductive member to which a potential is applied, from the conductive member by changing the potential so as to remove the interaction, and can be suitably selected according to the purpose.

The molecule-releasing means, for example, allows the molecules to be readily and efficiently released from the conductive member by electric repulsion by applying, to the conductive member to which a potential (e.g., a positive potential) is applied and the molecules are held by electrical attraction, a potential (e.g., a negative potential) opposite to the applied potential (a positive potential). More particularly, when the molecules are the polynucleotide that is a negatively charged negative ionic polymer, the application of a positive potential to the conductive member such as metal electrode causes the attraction and holding of the polynucleotide to the metal electrode by electronic interaction (Coulomb attraction). On the other hand, application of a negative potential to the metal electrode causes the release of the polynucleotide from the metal electrode by electric repulsion between the metal electrode and the polynucleotide. Thus, the molecules can be freely released from the conductive member at a desired time only by suitably changing the potential applied to the conductive member.

Preferred specific examples of the molecule-releasing means include a combination of at least one conductive member as one electrode, the second electrode as the counter electrode of the one electrode, and a power source that can apply a voltage, and a combination wherein the third electrode for controlling the potential between these electrodes are further provided in the combination.

In the molecule-releasing means, the voltage applied to the conductive member (electrode) is not specifically limited, and can be suitably selected according to the purpose. However, the voltage (the variation in the applied potential) is preferably at the level that does not damage or kill the cells as the transfer targets of the molecules, for example, preferably −0.8 V.

In comparison with publicly known methods such as electrotransfection, the voltage range is advantageous in enabling an adequately low voltage to be applied to the cells without damaging them. The voltage range is preferably adopted particularly when the conductive member and the second electrode arranged oppositely thereto are arranged to be stabbed into the same target (cell).

In the present invention, the molecule-releasing means is preferably capable of releasing (controlling) the molecules which are mutually different from a plurality of the conductive members at different times. In such a case, the arrangement of the plurality of the conductive members, for example on the periphery of cells that are the transfer targets of the molecules allow the treatment, differentiation, and other processing of the cells. The molecule-releasing means may operate in a way that only a part of the plurality of the conductive members serves for releasing the molecules. In such a case, the plurality of the conductive members, for example, are left to be stabbed into mutually adjoining cells, and the molecules is released and transferred into only a part of these cells, thus allowing the investigation of the actions of the molecules, and also allowing the investigation of the influence between the mutually adjoining cells.

They can be carried out by arranging the plurality of the conductive members on a passive matrix or an active matrix. A wiring structure in which the plurality of the conductive members is arranged on a passive matrix or an active matrix allows only the conductive member at desired position to be controlled at any time.

In the present invention, the molecule-releasing apparatus may comprise, for example, monitoring means as the other means. The monitoring means allows the transfer targets of the molecules such as cells to be observed, and makes it possible to safely, efficiently, and automatically treat and differentiate the cells.

The molecule-releasing apparatus of the present invention can be suitably used in various fields, and can be suitably used particularly as an apparatus for gene therapy, diagnosis of genetic diseases, and gene transfer, and as an analyzer using gene transfer.

(Molecule-Releasing Method)

The molecule-releasing method of the present invention includes at least a step of releasing molecules electrically attracted to two or more of the conductive members from the conductive members by electric repulsion at different times, and further includes suitably selected other steps.

The molecule-releasing method of the present invention can be suitably carried out using a modification of the apparatus for releasing molecules of the present invention wherein the number of the conductive members is two or more, and the two or more conductive members can be controlled at different times.

The molecule-releasing process can be carried out by independently controlling each of the two or more conductive members (electrodes), specifically by independently changing (applying a voltage) (e.g., changing to a potential opposite to the applied potential) the potential applied to the conductive members (electrodes). The molecule-releasing process releases the molecules that have been electrically attracted to the conductive members from the conductive members by electric repulsion. At this moment, if the transfer targets of the molecules such as cells are arranged in proximity of the conductive members, or when the needle electrodes used as the conductive members are arranged to be stabbed into the targets such as cells, the molecules can be efficiently released or transferred to the cells.

The molecule-releasing method of the present invention can be suitably used in various fields, and can be suitably used particularly for gene therapy, diagnosis of genetic diseases, gene transfer, and detailed observation using gene transfer.

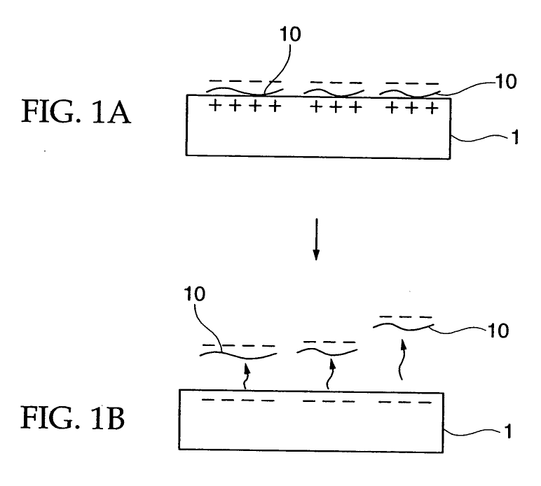

The principle of releasing molecules in the molecule-releasing apparatus of the present invention will described with reference to the drawings. As shown in FIGS. 1A and 1B, an example of the molecule-releasing apparatus of the present invention includes at least DNA molecules 10 as the molecules and a metal electrode 1 as the conductive member that attracts the molecules by electrical interaction and thereby holds the molecules. Above the metal electrode 1, the second electrode is provided (not shown). The metal electrode 1 and the second electrode are connected to a power source (not shown), and arranged to be immersed in a conductive liquid. The molecule-releasing means is comprised of the metal electrode 1, the second electrode, and the power source.

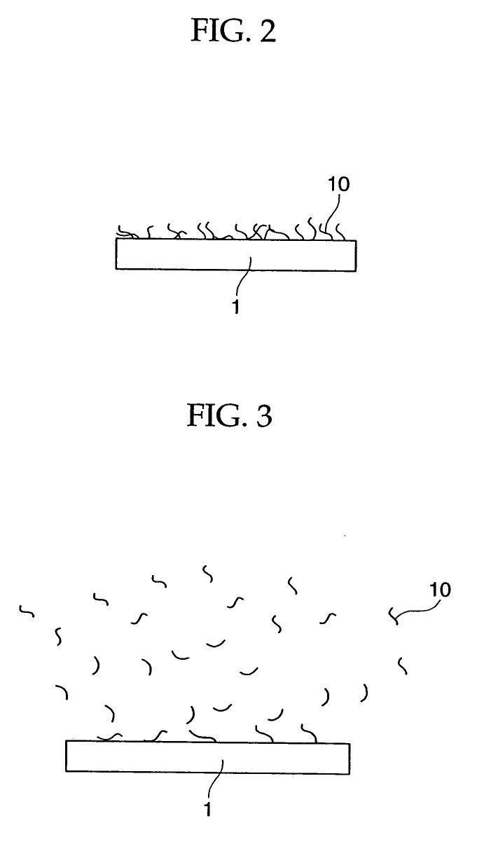

When the power source as the molecule-releasing means is in operation to apply a positive potential to the metal electrode 1, as shown in FIGS. 1A and 2, a negatively charged DNA molecules are attracted and held to the positively charged metal electrode 101 to which a positive potential is applied by electric interaction (Coulomb attraction). On the other hand, when the power source is put in operation to slightly apply a potential opposite to the positive potential (a negative potential) to the metal electrode 1 (a voltage (−800 mV) is slightly applied), as shown in FIGS. 1B and 3, the negatively charged DNA molecules 10 are released by electric interaction from the negatively charged metal electrode 1 due to the application of a negative potential by the power source.

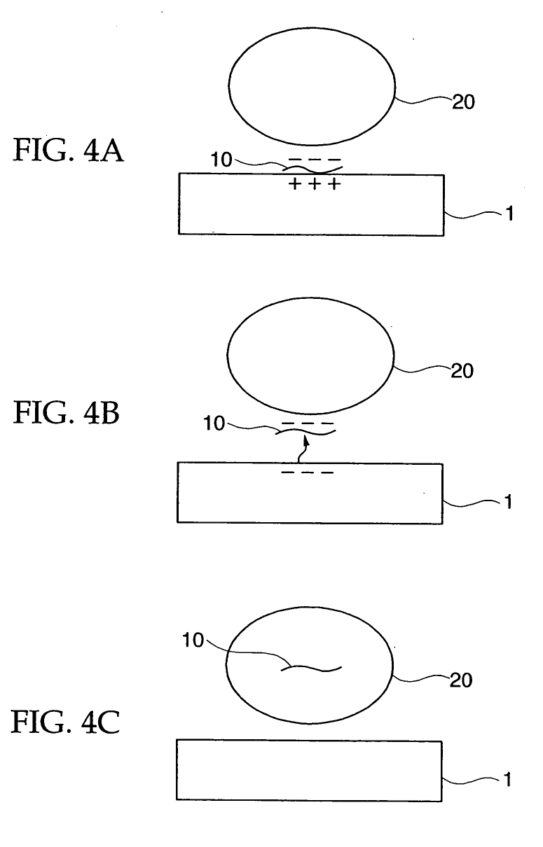

At the time, as shown in FIG. 4A, if a cell 20, which is surface treated (added with liposome) for easy transfer of the DNA molecule 10, is arranged in proximity of the metal electrode 1 as the transfer target of the molecule, as shown in FIG. 4B, the DNA molecule 10 released from the metal electrode 1 approaches the cell 20, and as shown in FIG. 4C, the DNA molecule 10 is transferred to the cell 20. In such a case, different from cases with an electron gun, the microinjection method, and electrotransfection, the molecule can be transferred into the cell 20 only by applying a slight voltage, thus the cell 20 is not damaged, and a desired molecule such as a DNA molecule can be transferred (introduced) into the target cell at a desired time. Therefore, the molecule-releasing apparatus of the present invention is excellent in safety and is suitably applicable to gene therapy or other applications.

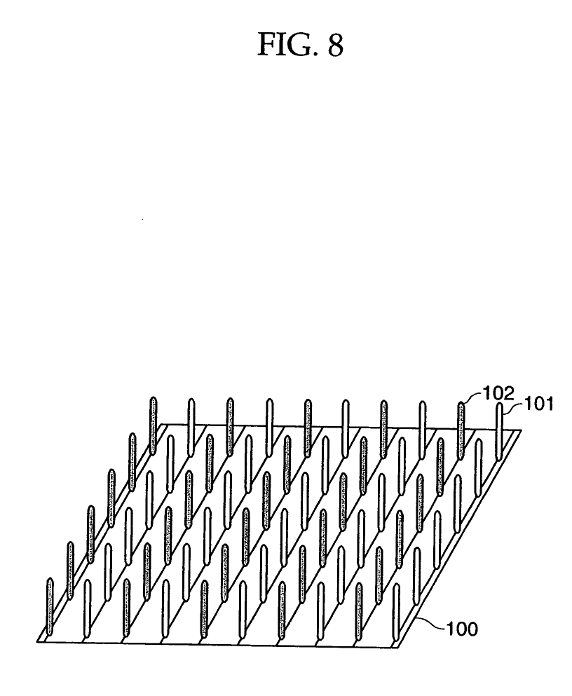

Next, another example of the molecule-releasing apparatus of the present invention will be described with reference to the drawings. As shown in FIG. 8, another example of the molecule-releasing apparatus of the present invention includes needle electrodes 101 as the conductive members for attracting and holding DNA molecules (not shown) as the molecules by electric interaction; second electrodes 102; bed-of-nails substrate 100 on which the needle electrodes 101 and the second electrodes 102 are arranged alternatively in order (arrangement interval: 5 μm) as to face each other. The needle electrodes 101 and the second electrodes 102 are connected to a power source (not shown), and arranged to be immersed in a conductive liquid. The molecule-releasing means is comprised of the needle electrodes 101, second electrodes 102, and power source.

When the power source as the molecule-releasing means is in operation to apply a positive potential to the needle electrodes 101, a negatively charged DNA molecules are attracted and held to the positively charged metal electrode 101 to which a positive potential is applied by electric interaction (Coulomb attraction).

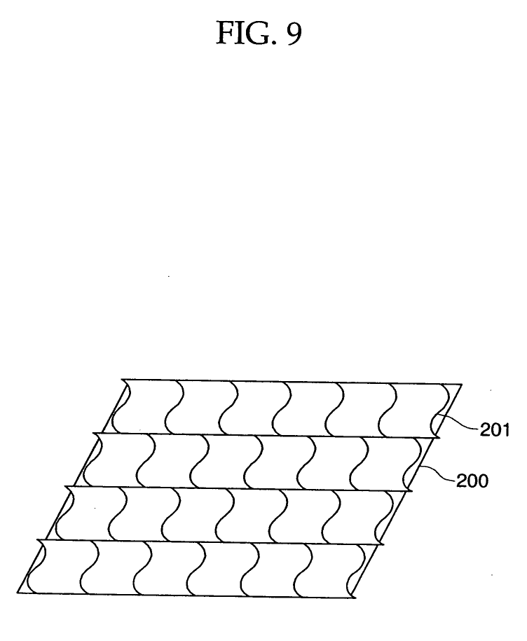

As shown in FIG. 9, the cells 201 as the transfer targets of the molecules are monolayer cultured on a plate (culture plate) 200 for previously obtaining a monolayer cultured layer in a confluent state. The intervals of the cells in the monolayer cultured layer are 10 μm or more, thus at least two needle electrodes can be stabbed into one cell based on calculation.

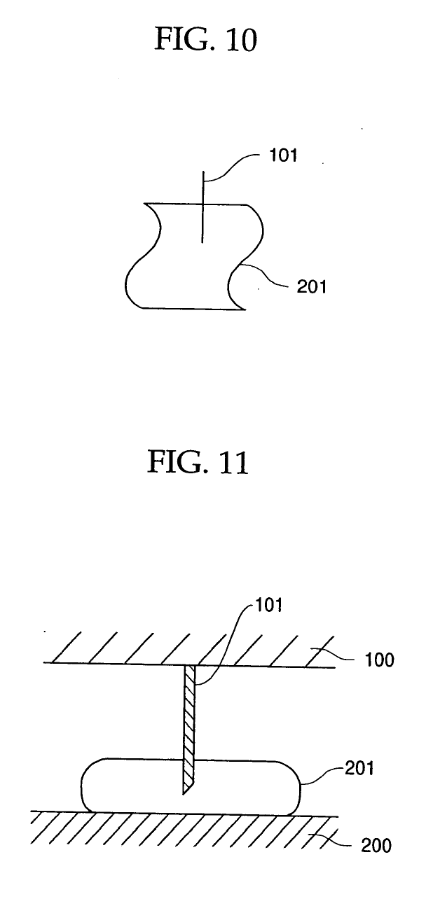

As shown in FIGS. 10 and 11, one of the tips of the bed-of-nails electrode substrate 100, on which the needle electrodes 101 electrically attracting and holding DNA molecules to their tips and the second electrodes 102 are arranged alternatively in lines to face each other, is arranged to be stabbed into a cell 201 on the monolayer cultured layer. In this state, the power source is put in operation for slightly applying a potential (negative potential) opposite to the positive potential that has been applied to the needle electrodes 101 (slightly applying voltage (−800 mV)). As the needle electrodes 101 on the bed-of-nails electrode substrate 100 each are connected as an active matrix electrode (or a passive matrix electrode), a potential on the needle electrodes 101 in desired positions can be changed (a voltage can be applied to) at a desired time and to a desired degree. Then, the negatively charged DNA molecules are released from the negatively charged needle electrode 101 by electrical interaction (Coulomb attraction), and transferred into the cell 201. In such a case, different from cases with an electron gun, the microinjection method, and electrotransfection, the molecule can be transferred into the cells 201 only by applying a slight voltage. Thus, desired molecules such as DNA molecules can be efficiently transferred into the cells 201 at a desired time without damaging the cells 201.

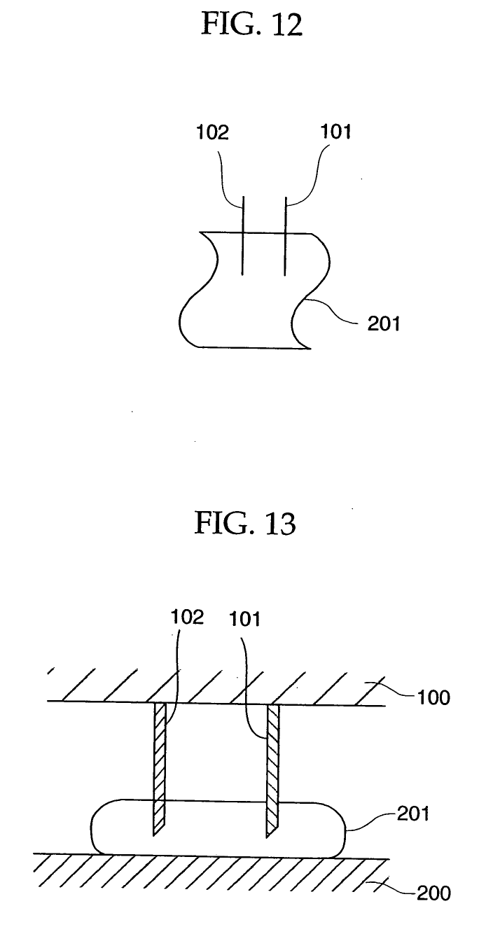

As shown in FIGS. 12 and 13, a voltage may be applied between a needle electrode 101 and a second electrode 102 arranged to be stabbed into a cell 201. In such a case, the potential to be applied can be controlled within a desired range to control the potential difference (applied voltage) for not loading too high a potential (not applying too high a voltage) to the cell 201, and this is preferred in preventing the cell 201 from being subjected to severe damages to be killed, etc.

The present invention will be illustrated in further detail with reference to several examples below, which are not intended to limit the scope of the present invention.

### EXAMPLE 1

Single chain polynucleotides (12 bases in the example) having a (CH₂)₃SS(CH₂)₃OH group at their 3′ terminal were synthesized as the molecules.

As shown in FIG. 2, the molecules were allowed to react with a polished circular metal electrode 1 having a diameter of 7 mm (where the electrode was a gold electrode) for 24 hours at room temperature. Then, the metal electrode 1 and the oppositely arranged second electrode were immersed in an electrolyte, a direct-current electric field was applied between the two electrodes, and a positive potential was applied to the metal electrode 1 (gold electrode). Then, the negatively charged molecules were attracted and held to the positively charged metal electrode 1 (gold electrode) by electronic interaction (Coulomb attraction). At the moment, the number of the molecules that had been electronically attracted to the metal electrode 1 (gold electrode) was, referring to the description of J. Am. Chem. Soc. 1999, 121, 10803-10821, calculated at 1.8×10¹⁰.

After that, a direct-current electric field was applied between the two electrodes, and a negative potential (that had been subjected to a modulation such as a pulse modulation) was applied to the metal electrode 1 (gold electrode). Then, as shown in FIG. 3, the molecules (those not strongly binding to the metal electrode 1 via SS) were dissociated and released from the metal electrode 1 (gold electrode) by Coulomb repulsion.

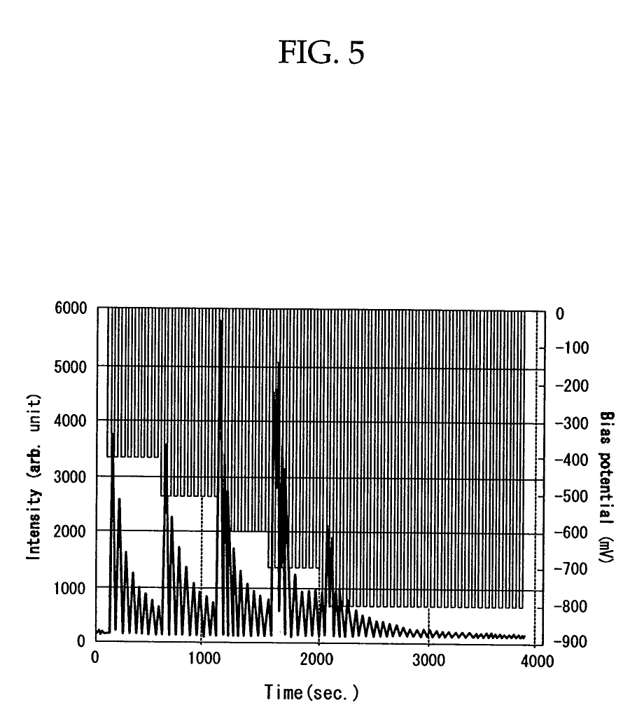

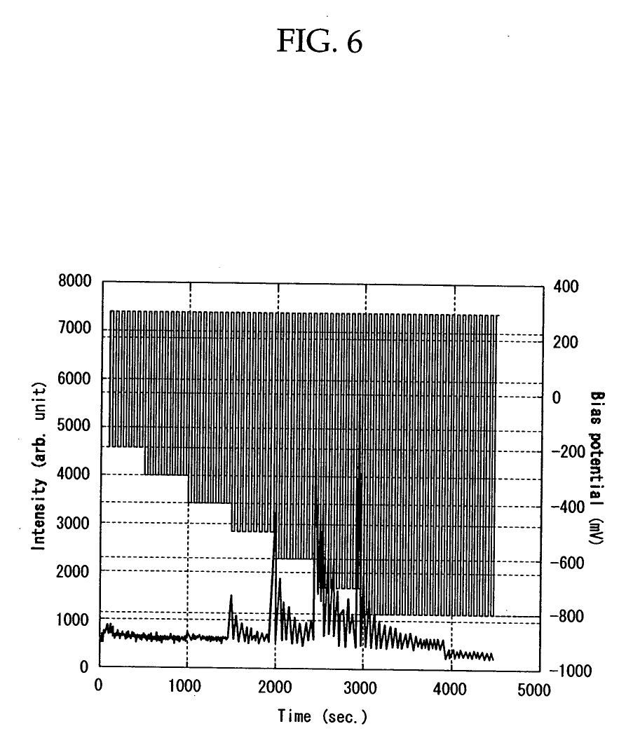

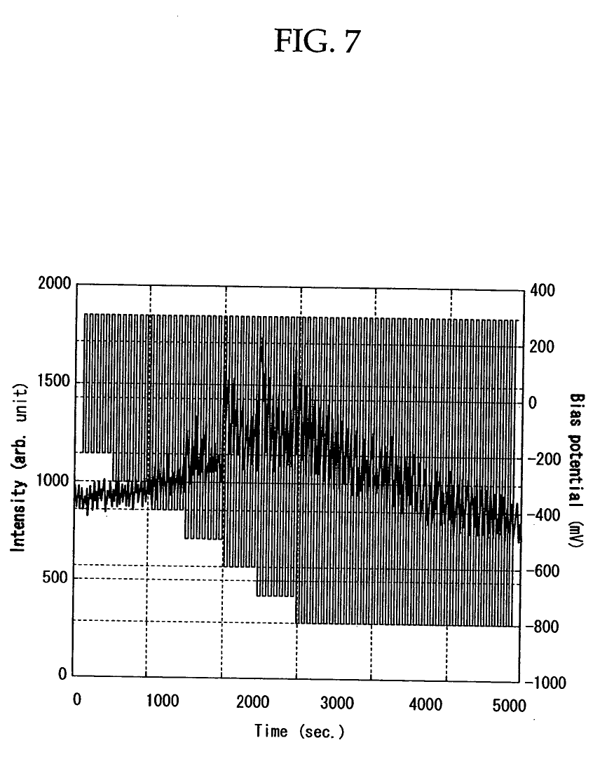

At the moment, as shown in FIG. 5, the DNA molecules 10 (those weakly interacting with the metal substrate 1) were released from the metal substrate 1 in relatively shorter times (the abscissa in FIG. 5 shows time (second)). As shown in FIG. 6, the DNA molecules 10 (excluding those weakly interacting with the metal substrate 1) were released from the metal substrate 1 in relatively longer times. As shown in FIG. 7, the complexes of DNA molecules and proteins were released from the metal substrate 1 in relatively long times.

### EXAMPLE 2

As shown in FIG. 8, as the conductive members and the second electrode, needle-shaped gold electrodes which are composed of carbon nanotubes of 0.2 μm in diameter and 3 μm in length with a gold-coated surface, were used. A bed-of-nails substrate 100 was fabricated by arranging a total of 10,000 of needle electrodes 101 as the conductive members and second electrodes 102 on a substrate (500 μm×500 μm) provided with an active matrix addressed electrical wiring so that lines of the needle electrodes and lines of the second electrodes mutually face and are alternatively arrayed (arrangement interval: 5 μm). Thus, the potential on the needle electrodes 101 and the second electrodes 102 in desired points on the bed-of-nails electrode substrate 100 could be changed (a voltage can be applied) at a desired time and to a desired degree.

On the other hand, DNA 400-mer (400 bases) molecules labeled with a fluorescence (Cy3) were prepared using a fluorescent (Cy3) label primer by the PCR method.

The fluorescence (Cy3)-labeled DNA molecules as the molecules and the bed-of-nails electrode substrate 100 as the conductive member were allowed to react for 24 hours at room temperature, and a direct-current electrical field was applied between the needle electrodes 101 (gold electrodes) and the second electrodes 102 in a condition where the needle electrodes 101 (gold electrodes) and the second electrodes 102 that had been oppositely arranged to adjoin the needle electrodes 101 were immersed in an electrolyte. Then, the negatively charged molecules were attracted to the tips of the positively charged needle electrodes (gold electrodes) 101 by electrical interaction (Coulomb attraction).

After that, commercially available normal human dendritic cells 201 (manufactured by TAKARA BIO INC.) as the transfer target of the molecules were monolayer cultured on a plate (culture plate) 200 using a lymphocyte growth media (LGM-3 BulletKit manufactured by BioWhittaker Inc.), the culture medium was removed, and the monolayer cultured layer in a confluent state as shown in FIG. 9 was prepared.

As shown in FIGS. 10 and 11, the tips of the bed-of-nails electrode substrate 100, on which lines of the needle electrodes 101 electrically attracting and holding the DNA molecules to their tips and lines of the second electrodes 102 are arrayed alternatively so as to face mutually, were arranged to be stabbed into the cells 201 on the monolayer cultured layer. In the state, the power source was put in operation for applying a voltage of −0.8 V to the needle electrode 101 for 30 seconds with reference to the third electrode (Ag/AgCl). As the needle electrodes 101 on the bed-of-nails electrode substrate 100 each are connected as an active matrix electrode, a desired voltage can be applied to the needle electrodes 101 in desired positions at a desired time. Then, the negatively charged DNA molecules were released from the negatively charged needle electrodes 101 by electrical interaction (Coulomb repulsion), and transferred into the cells 201.

After that, the needle electrodes 101 on the bed-of-nails electrode substrate 100 were pulled out from the monolayer cultured layer, and the monolayer cultured layer in the range of 200 μm×200 μm was examined for the presence of the Cy3-derived fluorescence using a microplate reader. As a result, the fluorescence was recognized in all the cells within the range of 200 μm×200 μm, indicating that the molecules were highly efficiently transferred into all the cells.

A part of cells on the monolayer cultured layer to which the molecules had been transferred was peeled off from the culture plate 200, and the culture medium was reinjected into the monolayer cultured layer for culturing; the peeled part was covered with the cell layer again. This indicates that the cells into which the molecules were transferred were not damaged.

The present invention provides a molecule-releasing apparatus and a molecule-releasing method which can resolve conventional problems, can efficiently release or transfer various useful molecules, e.g., DNA molecules into targets such as cells without damaging them and can be safely applied for uses in gene therapy and other applications.

While the present invention has been described with reference to what are presently considered to be the preferred embodiments, it is to be understood that the invention is not limited to the disclosed embodiments. On the contrary, the invention is intended to cover various modifications and equivalent arrangements included within the spirit and scope of the appended claims. The scope of the following claims is to be accorded the broadest interpretation so as to encompass all such modifications and equivalent structures and functions.

1. A molecule-releasing apparatus comprising:
a releasing unit configured to release molecules,
wherein the releasing unit comprises at least one conductive member and releases molecules electrically interacting with the conductive member to which a potential is applied, from the conductive member by changing the potential so as to remove the interaction.

2. A molecule-releasing apparatus according to claim 1, wherein the releasing unit releases the molecules electrically interacting with the conductive member to which a potential is applied, from the conductive member by applying a potential opposite to the potential, to the conductive member so as to remove the interaction.

3. A molecule-releasing apparatus according to claim 1, wherein the conductive member is arranged in proximity of the transfer targets of molecules.

4. A molecule-releasing apparatus according to claim 1, wherein the conductive member is arranged to be stabbed into the transfer targets of molecules.

5. A molecule-releasing apparatus according to claim 3, wherein the targets are selected from cells and microcapsules.

6. A molecule-releasing apparatus according to claim 1, wherein the conductive member is provided at the tip of a fiber scope.

7. A molecule-releasing apparatus according to claim 1, wherein the at least one conductive member is at least one electrode.

8. A molecule-releasing apparatus according to claim 1, wherein the number of the at least one conductive member is two or more.

9. A molecule-releasing apparatus according to claim 8, wherein the operation of the two or more conductive members is independently controlled.

10. A molecule-releasing apparatus according to claim 7, wherein the at least one electrode is at least one needle electrode.

11. A molecule-releasing apparatus according to claim 10, wherein the needle electrode is 0.2 μm or less in diameter and 3 μm or more in length.

12. A molecule-releasing apparatus according to claim 10, wherein the needle electrode comprises at least one selected from the group consisting of metal, a conductive resin, and a conductive carbon.

13. A molecule-releasing apparatus according to claim 10, wherein the molecules are held to at least the tip of the needle electrode by electrical interaction.

14. A molecule-releasing apparatus according to claim 10, wherein the number of the at least one needle electrode is two or more, and one of the same molecules and different molecules are held to the two or more needle electrodes by electrical interaction.

15. A molecule-releasing apparatus according to claim 7, wherein the releasing unit further comprises at least one of second electrode.

16. A molecule-releasing apparatus according to claim 15, wherein the electrode is needle-shaped, and the second electrode and the needle electrode are arranged to be stabbed into the same target.

17. A molecule-releasing apparatus according to claim 7, wherein the releasing unit further comprises at least one of third electrode.

18. A molecule-releasing apparatus according to claim 1, wherein the releasing unit is capable of releasing molecules at different times.

19. A molecule-releasing apparatus according to claim 1, wherein the shape of the molecules is linear.

20. A molecule-releasing apparatus according to claim 1, wherein the molecules have electrical polarity.

21. A molecule-releasing apparatus according to claim 1, wherein the molecules are selected from ionic polymers.

22. A molecule-releasing apparatus according to claim 21, wherein the ionic polymers are at least one selected from a polynucleotide and its complexes.

23. A molecule-releasing apparatus according to claim 22, wherein the polynucleotide has at least six bases.

24. A molecule-releasing apparatus according to claim 3, further comprising a monitoring unit configured to monitor the state of the targets.

25. A molecule-releasing method, wherein molecules electrically attracted to two or more conductive members are released by electrical repulsion from the conductive members at different times.

26. A molecule-releasing method according to claim 25, wherein the conductive members are needle electrodes arranged to be stabbed into cells.
